(19) United States
(12) Patent Application Publication
(10) Pub. No.: US 2012/0276003 A1
(43) Pub. Date: Nov. 1, 2012

(54) COMPOUND CONTAINING AN OXYGEN-15, PREPARATION AND USE THEREOF, AND COMPOSITION COMPRISING THEREOF

(75) Inventors: Jun Zeng, Beijing (CN); Qiyin Sun, Beijing (CN)

(73) Assignee: Beijing Top Grade Medical Equipment Co., Ltd., Beijing (CN)

(21) Appl. No.: 13/244,507

(22) Filed: Sep. 25, 2011

(30) Foreign Application Priority Data
Apr. 29, 2011 (CN) 201110111147.4

(51) Int. Cl. A61K 51/00; C01B 13/00; G21G 1/00

(52) U.S. Cl. 424/1.61; 376/156; 423/249

(57) ABSTRACT

The present disclosure relates to a compound comprising an oxygen-15 and a process for preparation thereof. It also relates to the use of the compound in positron and/or other nuclide imaging and use of the compound in obtaining a perfusion and/or metabolic image in an animal and/or human body. A process for preparing the compound comprises irradiating a compound comprising oxygen via irradiation energy in the range of 10 MeV to 430 MeV. The oxygen atom in the compound may then be allowed to be converted to an oxygen-15 positron nuclide through a photonuclear reaction. Provided that the molecular structure of the irradiated compound is not disrupted, a compound comprising an oxygen-15 is prepared.

### RELATED APPLICATION

This application claims priority to Chinese Application No.: 201110111147.4, filed on Apr. 29, 2011, entitled “A COMPOUND CONTAINING AN OXYGEN-15, PREPARATION AND USE THEREOF, AND COMPOSITION COMPRISING THEREOF,” at least some of which may be incorporated herein.

### FIELD

The present disclosure relates to a compound comprising an oxygen-15 and preparation thereof. It also relates to the use of such compound in positron or other nuclide imaging and/or in the perfusion and/or metabolic studies of animal and/or human bodies. The disclosure also relates to a composition comprising is the compound.

### BACKGROUND

Nuclear medicine imaging techniques, such as positron emission tomography (hereinafter “PET”), digital scintillator detector (hereinafter “DS”) and single photon emission computed tomography (hereinafter “SPECT”), for example, are effective in the diagnosis of diseases including heart disease and cancer. These techniques involve administration of labeled tracers with a specific radioisotope (hereinafter “radiopharmaceutical”), followed by the detection of γ-rays emitted from the tracers. Nuclear medicine imaging techniques have been widely used in clinics because of their high specificity and sensitivity to diseases. Such techniques also generally provide a high degree of information about diseases, compared to other imaging techniques.

In recent years, a series of radioactive halogen-labeled compounds including [¹⁸F]1-amino-3-fluorocyclobutanecarboxylic acid (hereinafter “[¹⁸F]FACBC”) have been discovered and developed as radiopharmaceuticals, and their clinical application is under investigation (e.g., Japanese Patent Laid-open No. 2000-500442; Jonathan McConathy et al., “Improved synthesis of anti-[¹⁸F]FACBC: improved preparation of labeling precursor and automated radiosynthesis”, Applied Radiation and Isotopes, (Netherlands). 2003, 58, p. 657-666; and Timothy M. Shoup et al., “Synthesis and Evaluation of [¹⁸F]1-Amino-3-fluorocyclobutane-1-carboxylic Acid to Image Brain Tumors.”, The Journal of Nuclear Medicine, 1999, 40, p. 331-338). [¹⁸F]FACBC is, for example, considered to be effective as a diagnostic tracer for highly proliferative tumors because it has a property of being taken up specifically by an amino acid transporter.

The process for preparing [¹⁸F]FACBC comprises: providing 1-(N-(t-butoxycarbonyl)amino)-3-[((trifluoromethyl)-sulfonyl)oxy]-cyclobutane-1-carboxylic acid methyl ester as a labeling precursor; substituting the triflate group at position 3 of the precursor with a radioactive fluorine; and carrying out deprotection by subjecting the resulting compound to an acidic condition (e.g., Japanese Patent Laid-open No. 2000-500442; Jonathan McConathy et al., “Improved synthesis of anti-[¹⁸F]FACBC: improved preparation of labeling precursor and automated radiosynthesis”, Applied Radiation and Isotopes, (Netherlands). 2003, 58, p. 657-666; and Timothy M. Shoup et al., “Synthesis and Evaluation of [¹⁸F]1-Amino-3-fluorocyclobutane-1-carboxylic Acid to Image Brain Tumors.”, The Journal of Nuclear Medicine, 1999, 40, p. 331-338).

However, the cost of preparing a compound labeled with [¹⁸F] is high, and the compound labeled with [¹⁸F] is limited to specific compounds (e.g., the compound cannot be labeled until some groups in the compound are substituted by a fluorine or a carbon).

In addition, the process for preparing a labeled compound by traditional cyclotron is not a photonuclear reaction, and the compound is required to be labeled by a prepared isotope through a chemical method. The nuclear reaction formulas for preparing ¹⁵O (Oxygen-15) are ¹⁵N (p, n) ¹⁵O and ¹⁴N (d, n) ¹⁵O. Because ¹⁵O has a very short half-life (e.g., of about 2 minutes), it is almost impossible that a compound comprising an ¹⁵O isotope is chemically labeled.

### FIGURES

FIG. 1 illustrates a radioactive decay curve of ¹⁵O-water in comparison with a theoretical decay curve of ¹⁵O.

FIG. 2 illustrates a lung cancer perfusion image with ¹⁵O-water as is described with respect to example 1.

FIG. 3 illustrates a brain perfusion image with ¹⁵O-water as described with respect to example 1.

### DESCRIPTION

The disclosure relates to a compound comprising an oxygen-15 (e.g., also referred to as a labeled compound comprising an oxygen atom, or referred to as a compound labeled with an oxygen-15) and a process for preparation thereof. The disclosure also relates to the use of the compound in positron imaging and the use of the compound in obtaining a perfusion and/or metabolic image in animal and/or human bodies. The disclosure also relates to a composition comprising the compound.

The ¹⁶O of a compound is converted to an ¹⁵O positron isotope by irradiating the compound through a photonuclear reaction (e.g., ¹⁶O (gamma, n)¹⁵O) (e.g., via a high-energy radiation therapy accelerator).

According to one embodiment, a process for preparing a compound comprising an oxygen-15 is provided. The process comprises utilizing an irradiation energy in the range of 10 MeV to 430 MeV (e.g., generated by a high energy electron accelerator and/or a proton, a heavy ion or a neutron treatment device) to irradiate a compound comprising an oxygen atom (e.g., water). The process also comprises allowing the oxygen atom in the compound to convert to an oxygen-15 positron nuclide through a photonuclear reaction. Provided that the molecular structure of the compound is not disrupted, the compound comprising the oxygen-15 is prepared.

In one example, water can be treated using the aforementioned process to generate water comprising an ¹⁵O. When the water comprising the oxygen-15 is is injected into the body, perfusion and/or metabolic images of the ¹⁵O-water in the body can be obtained by imaging methods such as PET. Perfusion and/or metabolic information of ¹⁵O-water can be imaged for clinical diagnostic purposes and/or for biomedical research. It will be appreciated that other compounds comprising ¹⁶O can also be prepared using a similar process, so long as the compound structure is not destroyed during preparation. Water is a desirable compound to prepare because it is generally more available and cheaper than other drugs. Moreover, water comprising ¹⁵O is safe and easily acceptable by animals and/or humans.

In other words, the process involves a quick preparation of a “golden” perfusion agent, such as ¹⁵O-water, for example, with a photonuclear reaction to diagnose cardiovascular disease, cerebral disorder, cancer, etc. with PET imaging, for example.

According to another embodiment, the oxygen positron nuclide is ¹⁵O. An ¹⁵O oxygen positron nuclide can be generated by irradiating an ¹⁶O in a compound (e.g., water). The ¹⁶O of the compound can be converted to ¹⁵O until the irradiation energy reaches a predetermined level. Generally speaking, the ¹⁵O can be prepared without other sources of contamination of oxygen isotopes.

It will be appreciated that if the energy of the high-energy ray is increased slowly from 10 MeV (e.g., million electron volts), the conversion from ¹⁶O to ¹⁵O can be carried out.

By way of example, a compound comprising an oxygen-15 (e.g., especially ¹⁵O-water) can be prepared for clinical application.

According to another embodiment, a composition comprising a compound comprising an oxygen-15 that is yielded via one or more of the processes herein described is provided.

According to yet another embodiment, the composition further comprises a pharmaceutically acceptable carrier and/or excipient. Substances such as impurities comprised in the water can also be considered as an acceptable carrier and/or excipient. Generally, the purity of the water comprising ¹⁵O is desired to be high (e.g., the content of impurities in the water is preferably 0.1 mass % or less, more preferably 0.01 mass % or less, most preferably 0.001 mass % or less). The content of impurities in water may be qualified if the water is commercially available for clinical use.

In one embodiment, the compound comprising an oxygen-15 that is prepared using processes described herein is used in positron or other nuclide imaging. For example, the compound may be used to obtain a perfusion and/or a metabolic image in an animal and/or human body.

In another embodiment, the compound comprising the oxygen-15 may be used in preparing a tracer that is used before positron or other nuclide imaging.

The process and/or the labeled compound comprising the oxygen-15 has the following beneficial effects:

1. Using a photonuclear reaction model which is different from the present isotope-preparing process to generate ¹⁵O positron nuclides, and inexpensive water is preferably used as the raw material for preparing a “golden” perfusion tracer;

2. Different from the present labeling technique, no complicated labeling technique is required;

3. Not changing of the chemical structure of the water when labeling;

4. Carrying out positron imaging quickly after irradiation, operating easily and simply;

5. Short half life of nuclides such as ¹⁵O nuclide, low radioactive pollution after imaging, being friendly to the environment;

6. The present technique is used to provide the tracer for the positron imaging of PET and DS;

7. The present technique can be used in clinical studies and/or preclinical research and/or development of medicine, which can shorten the cycle of examination and development, and thus save on costs associated therewith.

The obtained compound comprising an oxygen isotope can be used with nuclear medicine devices such as PET, PET-CT, PET-MRI and PET-MRI-CT. PET refers to Positron Emission Tomography, CT refers to X-ray Computed Tomography, MRI refers to Magnetic Resonance Imaging, PET-CT refers to Positron Emission Tomography-Computed Tomography, PET-MRI refers to Positron Emission Tomography-Magnetic Resonance Imaging, PET-MRI-CT refers to Positron Emission Tomography-Magnetic Resonance Imaging-X-ray Computed Tomography.

By way of example the process of preparing ¹⁵O-water may comprise mounting 5.0 ml of commercially available water for clinical use in an ampule on a irradiation chamber and passing high energy photons with an energy of 10 MeV or more (e.g., preferably 30-50 MV of photons) through the ampule such that the water is irradiated by the high energy photons. Generally, the dosage of the irradiation depends upon the desired/required amount of energy.

Herein are provided several examples to further clarify the techniques and/or systems described herein.

### Example 1

The general procedure of example 1 is as follow:

2.5 g of water for injection, wherein the air is removed under negative pressure, is frozen to 0° C. Then the frozen water is placed in an exposure chamber of a high energy accelerator. After being irradiated by 50 MV of photons with a dosage of 3000 Gy (gray)/minute for 5 minutes, ¹⁶O of the water is converted into ¹⁵O having a radioactivity of about 2220.0 MBq. This radio-dosage is adequate for clinical perfusion studies of a brain, myocardium, and/or tumors.

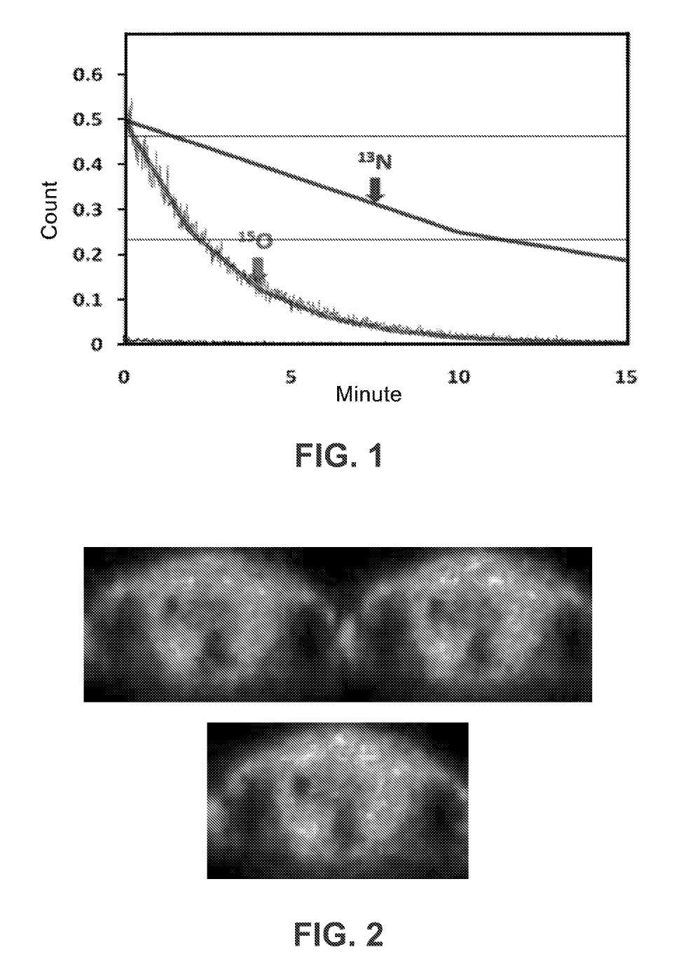

It is found that the water comprising ¹⁵O can be prepared using such a process. FIG. 1 illustrates a radioactive decay curve of ¹⁵O-water in comparison with a theoretical decay curve of ¹⁵O. The measured radioactive decay curve of ¹⁵O-water is fully in accordance with the theoretical decay curve of ¹⁵O, which indicates that the ¹⁵O-water can be prepared using the processes herein described.

FIG. 2 illustrates a PET perfusion image of ¹⁵O-water in a patient with a lung cancer.

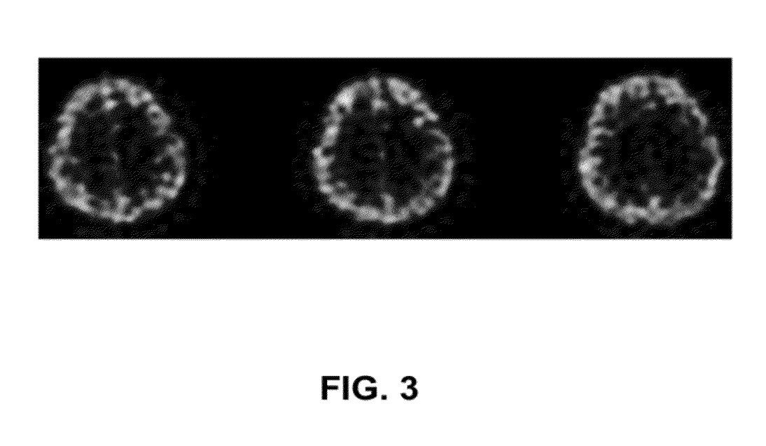

FIG. 3 illustrates a brain perfusion image with ¹⁵O-water in a patient.

From FIG. 2 and FIG. 3, it can be seen that the water comprising ¹⁵O prepared as described herein can be used for PET. ¹⁵O-water preparation, and diagnosis of diseases with a “golden” perfusion tracer, may have decreased cost relative to other ¹⁵O-water preparation techniques. For example, ¹⁵O-water prepared by exposing water to high-energy photons can cost RMB 1 yuan, whereas preparing ¹⁵O-water by traditional cyclotron can cost RMB 80,000 yuan.

### Example 2

The general procedure of example 2 is as follow:

2.5 g of water for injection, wherein the air is removed under negative pressure, is frozen to 0° C. The frozen water is then placed in an exposure is chamber of a high energy accelerator and irradiated by a ray of 50 MV with a dosage of 3000 Gy (gray)/minute for 5 minutes. The subsequent acts are the same as that of example 1. The same experimental effects as that of example 1 are obtained in example 2. The obtained PET labeling diagram is similar, and it is difficult to find differences between the effects of example 2 and that of example 1 with unaided eyes.

### Example 3

The general procedure of example 3 is as follow:

2.5 g of water for injection, wherein the air is removed under negative pressure, is frozen to 0° C. The frozen water is then placed in an exposure chamber of a high energy accelerator and irradiated by a ray of 49 MeV with the dosage of 3000 Gy (gray)/minute for 5 minutes. The subsequent acts are the same as that of example 1. The same experimental effects as that of example 1 are obtained in example 3. The obtained PET labeling diagram is similar, and it is difficult to find differences between the effects of example 3 and that of example 1 with unaided eyes.

Academically, ¹⁵O-water is a gold standard for a perfusion imaging agent, as the one-time through uptake rate is almost 100%. The molecular structure of water (H₂O) is very simple. However, the processes for preparing water comprising ¹⁵O by the traditional PET molecular imaging techniques are very complicated and expensive (e.g., with the cost of preparing the water comprising ¹⁵O being about RMB 80,000 yuan). Therefore, alternatives to ¹⁵O-water have been developed, such as ¹³N ammonia (heart and brain perfusion) and 99 mTc-MIBI (myocardial perfusion). These drugs are now widely used in clinical for the diagnosis of coronary heart disease and cerebrovascular disease. However, the one-time through uptake rate of such drugs is merely 70%-75%, and the diagnostic effect of these drugs is not better than that of the ¹⁵O-water.

It will be appreciated that there are two important differences over prior techniques. First, is the process for preparing the ¹⁵O-water by high energy photons and utilizing a photonuclear reaction (e.g. ¹⁶O(γ, n) ¹⁵O). Second is the preparation of ¹⁵O-compounds (e.g., ¹⁵O-water) without a chemical labeling procedure. For example, water comprising ¹⁵O may be prepared utilizing the photonuclear reaction instead of ¹⁴N(d, n)¹⁵O or ¹⁵(p, n)¹⁵O.

The process for preparing ¹⁵O-water by high energy photons is simple, rapid, and low cost.

What is claimed is:

1. A process, comprising:
utilizing irradiation energy in the range of 10 MeV to 430 MeV generated by at least one of a high energy electron accelerator, a proton treatment device, a heavy ion treatment device, or a neutron treatment device to irradiate a compound comprising an oxygen atom; and
allowing the oxygen atom to convert to an oxygen-15 positron nuclide through a photonuclear reaction, the molecular structure of the irradiated compound not disrupted, thereby preparing a compound comprising an oxygen-15.

2. The process of claim 1, comprising using the compound comprising the oxygen-15 in nuclide imaging.

3. The process of claim 1, comprising using the compound comprising the oxygen-15 to obtain at least one of a perfusion image or a metabolic image of at least one of an animal or a human body.

4. The process of claim 1, comprising using the compound comprising the oxygen-15 in preparing a tracer used before nuclide imaging.

5. The process of claim 1, comprising using the compound comprising the oxygen-15 in at least one of PET, PET-CT, PET-MRI, and PET-MRI-CT.

6. A compound comprising an oxygen-15 positron isotope prepared by irradiating an oxygen atom of a compound utilizing irradiation energy in the range of 10 MeV to 430 MeV and allowing the oxygen atom to convert to an oxygen-15 positron nuclide through a photonuclear reaction.

7. A composition comprising a compound comprising an oxygen-15 positron isotope prepared by irradiating an oxygen atom of a compound utilizing irradiation energy in the range of 10 MeV to 430 MeV and allowing the oxygen atom to convert to an oxygen-15 positron nuclide through a photonuclear reaction.

8. The composition of claim 7, comprising at least one of a pharmaceutically acceptable carrier or a pharmaceutically acceptable excipient.
